(19) United States
(12) Patent Application Publication
(10) Pub. No.: US 2017/0226441 A1
(43) Pub. Date: Aug. 10, 2017

(54) METHOD FOR PREPARING LOW-VISCOSITY LUBRICATING POLYOLEFINS

(71) Applicant: TOTAL MARKETING SERVICES, PUTEAUX (FR)

(72) Inventors: Marion COURTIADE, TERNAY (FR); Julien SANSON, LYON (FR); Alexandre WELLE, COURT-ST-ETIENNE (BE); Martine SLAWINSKI, NIVELLES (BE); Jeroen WASSENAAR, HUIZINGEN (BE)

(21) Appl. No.: 15/314,666

(22) PCT Filed: May 29, 2015
(86) PCT No.: PCT/EP2015/061956

§ 371 Date: Nov. 29, 2016

(30) Foreign Application Priority Data
May 30, 2014 (FR) 1454926

(51) Int. Cl. C10M 107/10; C07C 2/32; C07C 5/03; C10M 105/04

(52) U.S. Cl. CPC C10M 107/10; C10M 105/04; C07C 2/32; C07C 5/03; C10N 2240/10; C10N 2230/54; C10N 2230/10; C10N 2230/02; C10M 2205/0285; C10M 2203/0206; C10N 2240/04; C10N 2240/08; C10N 2050/10

(57) ABSTRACT

Disclosed is a method for preparing a low-viscosity oil including more than 50 wt % of 9-methyl-11-octyl-heneicosane. The method uses a specific metallocene catalyst and makes it possible to prepare a polyalphaolefin oil (PAO) in which the kinematic viscosity at 100° C., measured according to standard ASTM D445, ranges from 3 to 4 mm²/s⁻¹. The oil can be used as a high-performance lubricant for lubrication in the fields of engines, gears, brakes, hydraulic fluids, coolants and greases

The invention relates to a method for preparing a low viscosity oil comprising more than 50% by weight of 9-methyl-11-octyl-henicosane. This method applies a particular metallocene catalyst and allows preparation of a polyalphaolefin (PAO) oil, the kinematic viscosity of which at 100° C., measured according to the ASTM D445 standard, ranges from 3 to 4 mm²·s⁻¹. This oil may be used as a high performance lubricant for lubrication in the fields of engines, gears, brakes, hydraulic fluids, coolants, greases.

In the API classification of base oils, polyalphaolefins (PAO) are referenced as base oils of group IV. By means of a good compromise between viscosity, volatility and cold properties, these PAOs are increasingly used in high performance lubricant formulae. In particular, this best compromise is highly advantageous as compared with mineral bases of group III.

Generally, PAOs are synthesized from different olefinic monomers, in particular from C₆-C₁₄ monomers, by acid catalysis or in the presence of a metallocene catalyst.

Generally, for preparing low viscosity products the grades of which range from 2 to 10, acid catalysts are applied.

Methods for preparing PAOs by metallocene catalyst are known generally giving the possibility of resulting in high viscosity products, the kinematic viscosity of which at 100° C., as measured according to the ASTM D445 standard, ranges from 40 to 150 mm²·s⁻¹ (grades 40 to 150).

Moreover, the needs of high performance lubricants increase. In particular, because of the conditions of use with increasing severity, for example because of very high temperatures or mechanical stresses.

The oil drain interval and the reduction in the size of the lubrication systems also cause an increase in the need for high performance lubricants.

The energy efficiency and notably the improvement of the Fuel Eco (FE) of the lubricants or the reduction in the fuel consumption of engines, in particular vehicle engines, are increasingly significant goals and lead to the increasing use of high performance lubricants.

High performance lubricants should therefore have improved properties, in particular as regards the kinematic viscosity, the viscosity index, the volatility, the dynamic viscosity or the cold-pour point.

The thermal stability and the resistance to oxidation are also properties to be improved for high performance lubricants.

Reduced toxicity and good miscibility with other lubricants or other materials are also properties to be sought for high performance lubricants.

Moreover, improved methods for preparing PAOs should also be developed, in particular in order to improve the yield or the selectivity of these methods. Improvement of the catalytic activity should also be targeted.

The methods for preparing PAOs, should also give the possibility of recycling all or part of the secondary products stemming from oligomerization reactions.

The methods for preparing PAOs should also give the possibility of controlling the molecular mass as well as the polydispersity index and the distribution of the formed PAOs.

Improvement in the techniques for characterization of different products formed during the synthesis of PAOs is also to be sought, in particular during qualitative or quantitative analysis of the formed products.

WO-2013/055480 describes the preparation of useful PAOs as lubricants for a vehicle engine. This document describes a lubricant composition comprising such an oil associated with another base oil and with an additive improving the viscosity index. However, this patent application does not disclose 9-methyl-11-octyl-henicosane, nor its particular properties.

WO-2007/01973 describes the catalytic preparation of PAOs. This patent application describes the use of non-bridged metallocene catalysts. This document does not describe the preparation of 9-methyl-11-octyl-henicosane, nor its particular properties.

WO-2007/011459 describes PAOs obtained from the polymerization of C₅-C₂₄ olefins. This patent application does not disclose 9-methyl-11-octyl-henicosane, nor its particular properties.

WO-02/14384 describes a method for polymerization of olefins by metallocene catalysis. The described method only applies fluoro-cyclopentadienyl catalysts. This document does not describe the preparation of 9-methyl-11-octyl-henicosane.

Therefore there exists a need for preparing high performance lubricants giving the possibility of providing a solution to all or part of the problems of lubricants or of the methods for preparing lubricants of the state of the art.

Thus, the invention provides a method for preparing an oil with a kinematic viscosity at 100° C., measured according to the ASTM D445 standard, ranging from 3 to 4 mm²·s⁻¹, comprising more than 50% by weight of a 1-decene trimer of formula (I),

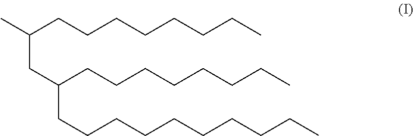 [SMILES: CCCCCCCCCCC(CCCCCCCC)CC(C)CCCCCCCC]

comprising
oligomerization of 1-decene in the presence of hydrogen (H₂), of a metallocene catalyst and of an activator compound or in the presence of hydrogen (H₂), of a metallocene catalyst, of an activator compound and of a co-activator compound;
catalytic hydrogenation of the oligomerization products in the presence of hydrogen (H₂) and of a hydrogenation catalyst;
separation by distillation at low pressure of the fraction of trimers comprising more than 50% by weight of the 1-decene trimer of formula (I).

Preferably, the method according to the invention comprises
oligomerization of 1-decene in the presence of hydrogen (H₂), of a metallocene catalyst, of an activator compound and of a co-activator compound;
catalytic hydrogenation of the oligomerization products in the presence of hydrogen (H₂) and of a catalyst selected from among a hydrogenation catalyst and a hydrogenation catalyst comprising palladium;
separation by distillation at low pressure of the fraction of trimers comprising more than 50% by weight of the 1-decene trimer of formula (I)

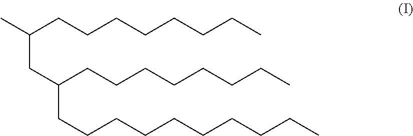 [SMILES: CCCCCCCCCCC(CCCCCCCC)CC(C)CCCCCCCC]

For the method according to the invention, the oligomerization of 1-decene is carried out in the presence of hydrogen (H₂). Thus, the presence of hydrogen during the oligomerization of 1-decene gives the possibility of obtaining an oil comprising more than 50% by weight of the 1-decene trimer of formula (I), the properties of which are of particular interest.

Preferably, the oligomerization of 1-decene is achieved in the presence of a metallocene catalyst which is a racemic compound of formula (II)

L(Q¹)(Q²)MR¹R² (II)
wherein
   M represents a transition metal selected from among titanium, zirconium, hafnium, and vanadium or represents zirconium;
   Q¹ and Q², either substituted or non-substituted, independently represent a tetrahydroindenyl cyclic group or Q¹ and Q² independently represent a tetrahydroindenyl cyclic group and are bound in order to form a polycyclic structure;
   L represents a divalent C₁-C₂₀ alkyl group bridging Q¹ and Q² or L represents a group selected from among methylene (—CH₂—), ethylene (—CH₂—CH₂—), methylmethylene (—CH(CH₃)—), 1-methyl-ethylene (—CH(CH₃)—CH₂—), n-propylene (—CH₂—CH₂—CH₂—), 2-methylpropylene (—CH₂—CH(CH₃)—CH₂—), 3-methylpropylene (—CH₂—CH₂—CH(CH₃)—), n-butylene (—CH₂—CH₂—CH₂—CH₂—), 2-methylbutylene (—CH₂—CH(CH₃)—CH₂—CH₂—), 4-methylbutylene (—CH₂—CH₂—CH₂—CH(CH₃)—), pentylene and isomers thereof, hexylene and isomers thereof, heptylene and isomers thereof, octylene and isomers thereof, nonylene and isomers thereof, decylene and isomers thereof, undecylene and isomers thereof, dodecylene and isomers thereof;
   R¹ and R², either substituted or non-substituted, independently represent an atom or a group selected from among hydrogen, halogens (such as Cl and I), alkyls (such as Me, Et, nPr, iPr), alkenyls, alkynyls, halogenoalkyls, halogenoalkenyls, halogenoalkynyls, silylalkyls, silylalkenyls, silylalkynyls, germylalkyl, germylalkenyl, germylalkynyl; or R¹ and R² form with M a metallocycle comprising from 3 to 20 carbon atoms.

More preferably, the metallocene catalyst is a racemic compound of formula (II) wherein
M represents zirconium;
Q¹ and Q², either substituted or non-substituted, independently represent a tetrahydroindenyl cyclic group;
L represents a group selected from among methylene (—CH₂—), ethylene (—CH₂—CH₂—), methylmethylene (—CH(CH₃)—), 1-methyl-ethylene (—CH(CH₃)—CH₂—), n-propylene (—CH₂—CH₂—CH₂—), 2-methylpropylene (—CH₂—CH(CH₃)—CH₂—), 3-methylpropylene (—CH₂—CH₂—CH(CH₃)—), n-butylene (—CH₂—CH₂—CH₂—CH₂—), 2-methylbutylene (—CH₂—CH(CH₃)—CH₂—CH₂—), 4-methylbutylene (—CH₂—CH₂—CH₂—CH(CH₃)—), pentylene and isomers thereof, hexylene and isomers thereof, heptylene and isomers thereof, octylene and isomers thereof, nonylene and isomers thereof, decylene and isomers thereof, undecylene and isomers thereof, dodecylene and isomers thereof;
R¹ and R², either substituted or non-substituted, independently represent a halogen atom, such as Cl and I, or an alkyl group, such as Me, Et, nPr, iPr.

Still more preferably, the metallocene catalyst is selected from among rac-ethylene bis(tetrahydroindenyl) zirconium dimethyl and rac-ethylene bis(tetrahydroindenyl)zirconium dichloride, in particular rac-ethylene bis(tetrahydroindenyl) zirconium dimethyl.

For the method according to the invention, the catalyst is applied in a form activated for oligomerization of 1-decene. Thus, the method according to the invention applies an activator compound during the oligomerization of 1-decene.

Advantageously, the activator compound is selected from among an alumoxane, an ionic activator and mixtures thereof.

Preferably for the method according to the invention, the alumoxane is an oligomeric compound comprising residues of formula —Al(R)—O— wherein R independently represents a C₁-C₂₀ cyclic or linear alkyl group. Preferably, the alumoxane is selected from among methylalumoxane, modified methylalumoxane, ethylalumoxane, isobutylalumoxane and mixtures thereof.

Also preferably, the alumoxane is applied in a alumoxane/catalyst molar ratio ranging from 1 to 10,000, preferably ranging from 10 to 3,000 and more preferentially from 100 to 1,500.

Preferably for the method according to the invention, the activator compound is an ionic activator. The ionic activator may be selected from dimethylanilinium tetrakis-(perfluorophenyl)borate (DMAB), triphenylcarbonium tetrakis-(perfluorophenyl)borate, dimethylanilinium tetrakis-(perfluorophenyl)aluminate and mixtures thereof. More preferably, the ionic activator is dimethylanilinium tetrakis-(perfluorophenyl)borate (DMAB).

Also preferably, the ionic activator is applied in an ionic activator/catalyst molar ratio ranging from 0.5 to 4, preferably from 0.8 to 1.2.

During the oligomerization of 1-decene, the method according to the invention applies an activator compound. It may also be advantageous to apply a co-activator compound, in particular during the use of an ionic activator.

Preferably, the co-activator compound is a trialkylaluminium derivative. More preferably, the co-activator compound is selected from among tri-ethyl aluminium (TEAL), tri-iso-butyl aluminium (TIBAL), tri-methyl aluminium (TMA), methyl-methyl-ethyl aluminium (MMEAL) and tri-n-octyl aluminium. Advantageously, tri-iso-butyl aluminium (TIBAL) is applied as a dispersion which may range from 10 to 60% by mass.

Also preferably, the co-activator compound is applied in a co-activator compound/catalyst molar ratio ranging from 10 to 1,000, preferably from 20 to 200.

Generally, the metallocene catalyst is activated by combining the metallocene catalyst and the activator compound, simultaneously or sequentially, according to pre-established time intervals. The combination of the metallocene catalyst and of the activator compound may be produced in the presence or in the absence of 1-decene and of hydrogen (H₂). During the oligomerization of 1-decene, the co-activator compound may be introduced with the activator compound. Preferably, the activated catalyst is prepared in advance and then introduced into the oligomerization reactor with 1-decene and hydrogen (H₂).

Advantageously, the metallocene catalyst and the activator compound, optionally in the presence of a co-activator compound, are put into contact at a pressure of 1 bar and at a temperature of 20° C.

Advantageously, the oligomerization of 1-decene is achieved within a period ranging from 2 to 300 mins. Preferably, the oligomerization period ranges from 5 to 180 mins, in particular from 30 to 140 mins.

Also advantageously, the oligomerization of 1-decene is achieved in the presence of hydrogen (H₂) at a partial pressure ranging from 0.1 to 20 bars. Preferably, the partial pressure of hydrogen (H₂) ranges from 1 to 6 bars.

Also advantageously, the oligomerization is achieved in a hydrogen/1-decene mass ratio greater than 100 ppm or less than 600 ppm. Preferably, this ratio is comprised between 100 and 600 ppm.

Also advantageously, the oligomerization of 1-decene is achieved at a temperature ranging from 50 to 200° C., preferably from 70 to 160° C. More preferably, the temperature during oligomerization of 1-decene ranges from 80 to 150° C. and still more preferably from 90 to 140° C. or from 100 to 130° C.

The oligomerization of 1-decene may be achieved in 1-decene which is then used as a support for the reaction. The reaction is then advantageously conducted in the absence of a solvent.

Oligomerization of 1-decene may also be achieved in a solvent. Preferably, the solvent may be selected from among a linear or branched hydrocarbon, a cyclic or non-cyclic hydrocarbon, an alkylated aromatic compound and mixtures thereof. As preferred solvents for oligomerization of 1-decene, the use of a solvent is preferred, selected from among butanes, pentanes, hexanes, heptanes, octanes, cyclopentane, cyclohexane, methylcyclopentane, methylcyclohexane, methylcycloheptane, toluene, xylene and mixtures thereof.

After oligomerization of 1-decene, the method according to the invention applies catalytic hydrogenation of the oligomerization products. Catalytic hydrogenation of the oligomerization products is carried out in the presence of hydrogen (H₂) and of a hydrogenation catalyst.

Preferably, the hydrogenation catalyst is selected from among a derivative of palladium, a supported derivative of palladium, a derivative of palladium supported on alumina (for example on gamma-alumina), a derivative of nickel, a supported derivative of nickel, a derivative of nickel supported on kieselguhr, a derivative of platinum, a supported derivative of platinum, a cobalt-molybdenum derivative, a supported cobalt-molybdenum derivative.

More preferably, the hydrogenation catalyst comprises palladium. A particularly preferred hydrogenation catalyst comprises palladium supported on alumina (for example on gamma-alumina).

Also preferably, the hydrogen pressure (H₂) during the catalytic hydrogenation of the oligomerization products ranges from 5 to 50 bars, more preferably from 10 to 40 bars, in particular from 15 to 25 bars.

After the oligomerization of 1-decene and the catalytic hydrogenation of the oligomerization products, the method according to the invention comprises the separation by distillation at a reduced pressure of the fraction of trimers comprising more than 50% by weight of the 1-decene trimer of formula (I).

The separation by distillation is carried out at a reduced pressure. Advantageously, the separation by distillation is carried out according to the ASTM D2892 standard or according to the ASTM D5236 standard.

More advantageously, the separation is carried out in two steps, by distillation according to the ASTM D2892 standard and then by distillation according to the ASTM D5236 standard.

Preferably, during the separation by distillation according to the ASTM D2892 standard, the initial boiling point (IBP) is less than 370° C., preferably less than 375° C. The partial pressure is advantageously less than 0.67 mbars (0.5 mmHg).

The distillation according to the ASTM D2892 standard gives the possibility of separating the products for which the boiling point is less than these temperatures. Preferably, during the separation by distillation according to the ASTM D5236 standard, the initial boiling point (IBP) is comprised between 360 and 485° C., preferably between 370 and 480° C. or between 370 and 470° C. More preferably, the initial boiling point is comprised between 375 and 465° C. during the application of separation by distillation according to the ASTM D5236 standard. The partial pressure is advantageously less than 0.67 mbars (0.5 mmHg).

Preferably, the separation by distillation according to the ASTM D5236 standard gives the possibility of separating the fraction of trimers comprising more than 50% by weight of 1-decene trimer of formula (I).

Thus, the separation by distillation at reduced pressure gives the possibility of separating the fraction of trimers stemming from the oligomerization of 1-decene and then from hydrogenation of the oligomerization products. This fraction of trimers comprises more than 50% by weight of the 1-decene trimer of formula (I).

In addition to the oligomerization steps of 1-decene, of catalytic hydrogenation of the oligomerization products and separation by distillation at reduced pressure of the fraction of trimers comprising more than 50% by weight of 1-decene trimer of formula (I), the method according to the invention may advantageously comprise other steps. Thus, the method according to the invention may also combine all or part of the following steps:
preliminary preparation of 1-decene by catalytic oligomerization of ethylene;
deactivation of the catalyst after oligomerization of 1-decene or after the catalytic hydrogenation of the oligomerization products;
recycling the fraction of dimers of 1-decene (for example 9-methyl-nonadecane), separated by distillation at reduced pressure and oligomerization of this fraction of dimers of 1-decene, recycled with 1-decene, in the presence of hydrogen (H₂), of a metallocene catalyst and of an activator compound or in the presence of hydrogen (H₂), of a metallocene catalyst, of an activator compound and of a co-activator compound;
a final step for hydrogenation of the fraction of trimers comprising more than 50% by weight of 1-decene trimer of formula (I) in the presence of hydrogen (H₂) and of a catalyst selected from among a hydrogenation catalyst and a hydrogenation catalyst comprising palladium.

The preliminary preparation of 1-decene by catalytic oligomerization of ethylene is known as such. It may prove to be particularly advantageous in combination with the other steps of the method according to the invention. This preliminary preparation of 1-decene by catalytic oligomerization of ethylene notably gives the possibility of using more abundant sources of the initial substrate.

Moreover and preferably, once oligomerization of 1-decene is completed, the method according to the invention may comprise deactivation of the catalyst.

The deactivation of the oligomerization catalyst may be achieved after oligomerization of 1-decene or after catalytic hydrogenation of the oligomerization products. Preferably, the deactivation of the oligomerization catalyst is achieved after oligomerization of 1-decene and before catalytic hydrogenation of the oligomerization products.

Advantageously, the deactivation of the catalyst is achieved by action of air or water or by means of at least one alcohol or of a deactivation agent solution. Preferably, the deactivation of the catalyst is achieved by means of an alcohol, for example isopropanol.

Particularly advantageously, the method according to the invention may also comprise the recycling of the fraction of dimers of 1-decene which is separated by distillation at reduced pressure and then the oligomerization of this fraction of dimers of 1-decene recycled with 1-decene. Preferably, this recycled fraction of dimers of 1-decene comprises 9-methyl-nonadecane.

The oligomerization of this recycled fraction of dimers of 1-decene may then be achieved in the presence of hydrogen (H₂), of a metallocene catalyst and of an activator compound or else in the presence of hydrogen (H₂), of a metallocene catalyst, of an activator compound and of a co-activator compound.

The oligomerization of this recycled fraction of dimers of 1-decene may be achieved in the oligomerization reactor of 1-decene or else in one or several distinct reactors. Preferably, it is carried out in the oligomerization reactor of 1-decene and under the same conditions as this oligomerization of 1-decene.

In a particularly advantageous way, the recycling and then the oligomerization of this recycled fraction of dimers of 1-decene with 1-decene gives the possibility of improving the overall yield of the preparation method according to the invention and of thus producing a larger amount of oil according to the invention comprising more than 50% by weight of 9-methyl-11-octyl-henicosane.

Also advantageously, the method according to the invention may comprise a final step for hydrogenation of the fraction of trimers comprising more than 50% by weight of a trimer of 1-decene of formula (I). This final hydrogenation is carried out in the presence of hydrogen (H₂) and of a hydrogenation catalyst.

Preferably, the hydrogenation catalyst is selected from among a derivative of palladium, a supported derivative of palladium, a derivative of palladium supported on alumina (for example on gamma-alumina), a derivative of nickel, a supported derivative of nickel, a derivative of nickel supported on kieselguhr, a platinum derivative, a supported derivative of platinum, a cobalt-molybdenum derivative, a supported cobalt-molybdenum derivative. More preferably, the hydrogenation catalyst comprises palladium. A particularly preferred catalyst comprises palladium supported on alumina (for example on gamma-alumina).

The hydrogenation catalyst is advantageously identical with the hydrogenation catalyst applied during the hydrogenation of the oligomerization products of 1-decene.

Advantageously, during the final hydrogenation, the hydrogen pressure (H₂) ranges from 5 to 50 bars or from 10 to 40 bars, preferably from 15 to 25 bars.

Also advantageously, during the final hydrogenation, the hydrogenation period is comprised between 2 and 600 mins, preferably between 30 and 300 mins.

Advantageously, during the final hydrogenation, the temperature ranges from 50 to 200° C. or from 60 to 150° C. Preferably, the temperature ranges from 70 to 140° C. or from 80 to 120° C.

Preferably, the method according to the invention is a method for which
oligomerization of 1-decene is achieved within a period ranging from 2 to 300 mins or from 5 to 180 mins or from 30 to 140 mins; or
oligomerization of 1-decene is achieved in the presence of hydrogen (H₂) at a partial pressure ranging from 0.1 to 20 bars or from 1 to 6 bars; or
oligomerization is achieved in the presence of hydrogen (H₂) in a hydrogen/1-decene mass ratio greater than 100 ppm or less than 600 ppm or comprised between 100 and 600 ppm; or
oligomerization of 1-decene is achieved at a temperature ranging from 50 to 200° C. or from 70 to 160° C. or from 80 to 150° C. or from 90 to 140° C. or from 100 to 130° C.; or
the metallocene catalyst is a racemic compound of formula (II)

L(Q¹)(Q²)MR¹R² (II)
wherein
   M represents a transition metal selected from among titanium, zirconium, hafnium, and vanadium or represents zirconium;
   Q¹ and Q², either substituted or non-substituted, independently represent a tetrahydroindenyl cyclic group or Q¹ and Q² independently represent a tetrahydroindenyl cyclic group and are bound in order to form a polycyclic structure;
   L represents a divalent C₁-C₂₀-alkyl group bridging Q¹ and Q² or L represents a group selected from among methylene (—CH₂—), ethylene (—CH₂—CH₂—), methylmethylene (—CH(CH₃)—), 1-methyl-ethylene (—CH(CH₃)—CH₂—), n-propylene (—CH₂—CH₂—CH₂—), 2-methylpropylene (—CH₂—CH(CH₃)—CH₂—), 3-methylpropylene (—CH₂—CH₂—CH(CH₃)—), n-butylene (—CH₂—CH₂—CH₂—CH₂—), 2-methylbutylene (—CH₂—CH(CH₃)—CH₂—CH₂—), 4-methylbutylene (—CH₂—CH₂—CH₂—CH(CH₃)—), pentylene and isomers thereof, hexylene and isomers thereof, heptylene and isomers thereof, octylene and isomers thereof, nonylene and isomers thereof, decylene and isomers thereof, undecylene and isomers thereof, dodecylene and isomers thereof;
   R¹ and R², either substituted or non-substituted, independently represent an atom or a group selected from among hydrogen, halogens (such as Cl and I), alkyl (such as Me, Et, nPr, iPr), alkenyl, alkynyl, haloalkyl, haloalkenyl, haloalkynyl, silylalkyls, silylalkenyls, silylalkynyls, germylalkyl, germylalkenyl, germylalkynyl; or R¹ and R² form with M a metallocycle comprising from 3 to 20 carbon atoms; or
the metallocene catalyst is selected from among rac-ethylene bis(tetrahydroindenyl) zirconium dimethyl and rac-ethylene bis(tetrahydroindenyl)zirconium dichloride; or
oligomerization of 1-decene is achieved in a solvent selected from among a linear or branched hydrocarbon, a cyclic or non-cyclic hydrocarbon, an alkylated aromatic compound and mixtures thereof or in a solvent selected from among butanes, pentanes, hexanes, heptanes, octanes, cyclopentane, cyclohexane, methylcyclopentane, methylcyclohexane, methylcycloheptane, toluene, xylene and mixtures thereof; or
the activator compound is selected from among an ionic activator and an oligomeric compound comprising residues of formula —Al(R)—O— wherein R represents independently a cyclic or linear C₁-C₂₀ alkyl group; or the activator compound is selected from among methylalumoxane, modified methylalumoxane, ethylalumoxane, isobutylalumoxane and mixtures thereof; or the activator compound is selected from among dimethylanilinium tetrakis(perfluorophenyl)borate (DMAB), triphenylcarbonium tetrakis(perfluorophenyl)borate, dimethylanilinium tetrakis(perfluorophenyl)aluminate and mixtures thereof; or
the co-activator compound is a trialkylaluminium derivative or a compound selected from among tri-ethyl aluminium (TEAL), tri-iso-butyl aluminium (TIBAL), tri-methyl aluminium (TMA), methyl-methyl-ethyl aluminium (MMEAL) and tri-n-octyl aluminium; or
the deactivation of the catalyst is achieved by the action of air or water or by means of at least one alcohol or one deactivation agent solution; or
the hydrogen pressure (H₂) during the catalytic hydrogenation of the oligomerization products ranges from 5 to 50 bars or from 10 to 40 bars or from 15 to 25 bars; or
the hydrogenation catalyst is selected from a palladium derivative, a supported palladium derivative, a palladium derivative supported on alumina (for example on gamma-alumina), a derivative of nickel, a supported derivative of nickel, a derivative of nickel supported on kieselguhr, a derivative of platinum, a supported derivative of platinum, a cobalt-molybdenum derivative, a supported cobalt-molybdenum derivative; or
the hydrogen pressure (H₂) during the final hydrogenation of the majority fraction by weight of 1-decene trimer of formula (I) ranges from 5 to 50 bars or from 10 to 40 bars or from 15 to 25 bars; or
the hydrogenation period during the final hydrogenation is comprised between 2 and 600 mins or between 30 and 300 mins; or
the final hydrogenation finale is achieved at a temperature ranging from 50 to 200° C. or from 60 to 150° C. or from 70 to 140° C. or from 80 to 120° C.; or
the hydrogenation catalyst, during the final hydrogenation of the fraction of trimers comprising more than 50% by weight of the 1-decene trimer of formula (I), is selected from among a derivative of palladium, a supported derivative of palladium, a palladium derivative supported on alumina (for example on gamma-alumina), a derivative of nickel, a supported derivative of nickel, a supported derivative of nickel on kieselguhr, a derivative of platinum, a supported derivative of platinum, a cobalt-molybdenum derivative, a supported cobalt-molybdenum derivative.

More preferably, the method according to the invention is a method combining the whole of these characteristics.

Still more preferably, the method according to the invention is a method for which
oligomerization of 1-decene is achieved within a period ranging from 30 to 140 mins;
oligomerization of 1-decene is achieved in the presence of hydrogen (H₂) at a partial pressure ranging from 1 to 6 bars;
oligomerization of 1-decene is achieved in a hydrogen/1-decene mass ratio comprised between 100 and 600 ppm;
oligomerization of 1-decene is achieved at a temperature ranging from 100 to 130° C.;
the metallocene catalyst is selected from among rac-ethylene bis(tetrahydroindenyl) zirconium dimethyl and rac-ethylene bis(tetrahydroindenyl)zirconium dichloride;
oligomerization of 1-decene is achieved in a solvent selected from among butanes, pentanes, hexanes, heptanes, octanes, cyclopentane, cyclohexane, methylcyclopentane, methylcyclohexane, methylcycloheptane, toluene, xylene and mixtures thereof;
the activator compound is selected from among an ionic activator selected from among dimethylanilinium tetrakis(perfluorophenyl)borate, triphenylcarbonium tetrakis(perfluorophenyl)borate, dimethylanilinium tetrakis(perfluorophenyl)aluminate and mixtures thereof;
the co-activator compound is a compound selected from among tri-ethyl aluminium (TEAL), tri-iso-butyl aluminium (TIBAL), tri-methyl aluminium (TMA), methyl-methyl-ethyl aluminium (MMEAL) and tri-n-octyl aluminium;
the deactivation of the catalyst is achieved by means of at least one alcohol;
the hydrogen pressure (H₂) during the catalytic hydrogenation of the oligomerization products ranges from 15 to 25 bars;
the hydrogenation catalyst is a derivative of palladium supported on alumina (for example on gamma-alumina);
the hydrogen pressure (H₂) during the final hydrogenation of the majority fraction by weight of the 1-decene trimer of formula (I) ranges from 15 to 25 bars;
the hydrogenation period during the final hydrogenation is comprised between 30 and 300 mins;
the final hydrogenation is achieved at a temperature ranging from 80 to 120° C.;
the hydrogenation catalyst, during the final hydrogenation of the fraction of trimers comprising more than 50% by weight of 1-decene trimer of formula (I), is a derivative of palladium supported on alumina (for example on gamma-alumina).

Particularly advantageously, the method according to the invention may also comprise the whole or at least one of the following additional steps:
preliminary preparation of 1-decene by catalytic oligomerization of ethylene;
the deactivation of the catalyst after oligomerization of 1-decene and before the catalytic hydrogenation of the oligomerization products;
recycling the fraction of 1-decene dimers (for example 9-methyl-nonadecane), separated by distillation at reduced pressure and oligomerization of said fraction of 1-decene dimers recycled with 1-decene, in the presence of hydrogen (H₂), of a metallocene catalyst, of an activator compound and of a co-activator compound;
a final hydrogenation step of the fraction of trimers comprising more than 50% by weight of 1-decene trimer of formula (I) in the presence of hydrogen (H₂) and of a catalyst selected from among a hydrogenation catalyst and a hydrogenation catalyst comprising palladium supported on alumina (for example on gamma-alumina).

For the method according to the invention, the oligomerization may be conducted sequentially. The reagents and the catalytic system are then introduced into a reactor in order to react up to a certain conversion level, either total or partial. Next, generally, the catalyst is deactivated.

An example of a sequential method according to the invention may notably comprise the introduction of 1-decene, alone or in a solvent, into a stirred reactor. The reactor is then heated to the desired temperature and a determined amount of hydrogen (H₂) is introduced into the reactor. Once the reaction conditions are established, the metallocene catalyst is introduced into the reactor. The oligomerization level is controlled by adapting the concentration of catalyst and of 1-decene in the reactor. After a particular reaction time, the catalyst is deactivated.

The oligomerization may also be carried out in a semi-continuous mode. The reagents and the catalytic system are then introduced into the reactor simultaneously and continuously in order to maintain constant the ratio of the amounts of 1-decene with respect to the catalytic system. The reaction takes place up to a pre-established conversion level. Next the catalyst is generally deactivated.

The oligomerization may also be achieved in a continuous mode. The reagents and the catalytic system are then introduced into the reactor simultaneously and continuously in order to maintain constant the ratio of the amount of 1-decene with respect to the catalytic system. The reaction products are continuously separated from the reactor, for example a reactor for continuous reaction with stirring (CSTR, continuous stirred tank reactor).

After oligomerization, the oligomerization products are hydrogenated in the presence of hydrogen (H₂) and of a hydrogenation catalyst.

The method according to the invention allows preparation of an oil, for which the kinematic viscosity is particularly advantageous and ranges from 3 to 4 mm²·s⁻¹. More advantageously, the kinematic viscosity of this oil ranges from 3.2 to 3.8 mm²·s⁻¹. Preferably, the kinematic viscosity of the oil is 3.4 mm²·s⁻¹, 3.5 mm²·s⁻¹ or 3.6 mm²·s⁻¹.

Also advantageously, the method according to the invention allows preparation of an oil for which the viscosity index is greater than 120 or comprised between 120 and 140 or between 125 and 135. Preferably, the viscosity index of this oil is greater than or equal to 130. According to the invention, the viscosity index is generally computed according to the ASTM D2270 standard.

Also advantageously, the method according to the invention allows preparation of an oil for which the measured volatility according to the ASTM D6375 standard is less than 10.8% by mass. Preferably, the volatility of this oil is less than 10.5% by mass.

Also advantageously, the method according to the invention allows preparation of an oil for which the dynamic viscosity (CCS) at −35° C., measured according to the ASTM D5293 standard, is less than 900 mPa·s. Also advantageously, the dynamic viscosity of this oil is less than 800 mPa·s. According to the invention, the dynamic viscosity of the oil is measured on a rotary dynamic viscosimeter (CCS cold cranking simulator, simulator of cold starting).

Also advantageously, the method according to the invention allows preparation of an oil for which the average molecular mass ranges from 300 to 1,000 g/mol, preferably from 350 to 450 g/mol. According to the invention, the average molecular mass is generally computed according to the ASTM D2502 standard.

Also advantageously, the method according to the invention allows preparation of an oil for which the pour point is less than or equal to −50° C., preferably less than or equal to −55 or −57° C. According to the invention, the pour point is generally measured according to the EN ISO 3016 standard.

Advantageously, the invention provides a method for preparing an oil combining
(a) a kinematic viscosity at 100° C., measured according to the ASTM D445 standard ranging from 3.2 to 3.8 mm²·s⁻¹;
(b) a viscosity index greater than 120;
(c) a volatility measured according to the ASTM D6375 standard of less than 10.8% by mass; and
(d) a dynamic viscosity (CCS) at −35° C., measured according to the ASTM D5293 standard of less than 900 mPa·s.

Also advantageously, the method according to the invention allows preparation of an oil combining these properties (a) and (b); (a) and (c); (a) and (d); (b) and (c); (b) and (d); (c) and (d); (a), (b) and (c); (a), (b) and (d); (a), (c) and (d); (b), (c) and (d).

Preferably, the method according to the invention allows preparation of an oil combining
(a) a kinematic viscosity at 100° C., measured according to the ASTM D445 standard of 3.4 mm²·s⁻¹, 3.5 mm²·s⁻¹ or 3.6 mm²·s⁻¹;
(b) a viscosity index greater than or equal to 130;
(c) a volatility measured according to the ASTM D6375 standard of less than 10.5% by mass; and
(d) a dynamic viscosity (CCS) at −35° C., measured according to the ASTM D5293 standard of less than 900 mPa·s.

Also preferably, the invention allows preparation of an oil combining these properties (a) and (b); (a) and (c); (a) and (d); (b) and (c); (b) and (d); (c) and (d); (a), (b) and (c); (a), (b) and (d); (a), (c) and (d); (b), (c) and (d).

Advantageously, the method according to the invention allows preparation of an oil comprising at least 65% by weight of 1-decene trimer of formula (I) or at least 70% by weight of a 1-decene trimer of formula (I). More advantageously, the oil prepared according to the invention comprises at least 80% by weight of 1-decene trimer of formula (I) or at least 90% by weight of 1-decene trimer of formula (I).

Preferably, the method according to the invention allows preparation of an oil comprising from 50 to 99% by weight of 1-decene trimer of formula (I). More preferably, the oil prepared according to the invention comprises from 60 to 90% by weight of 1-decene trimer of formula (I). Still more preferably, the oil prepared according to the invention comprises from 70 to 90% by weight of 1-decene trimer of formula (I).

Also preferably, the method according to the invention allows preparation of an oil comprising from 60 to 95% by weight, from 60 to 80% by weight, from 70 to 95% by weight, from 70 to 80% by weight, from 75 to 95% by weight or from 75 to 80% by weight of 1-decene trimer of formula (I).

In addition to the 1-decene trimer of formula (I), the oil prepared according to the invention may comprise other oligomers stemming from oligomerization of 1-decene. Thus, this oil may comprise at least one other saturated oligomer of 1-decene. Preferably, this other saturated oligomer of 1-decene may be selected from among the other saturated trimers of 1-decene. It may also be selected from among a wider group of saturated oligomers comprising dimers of 1-decene, the other trimers of 1-decene, the tetramers of 1-decene, the pentamers of 1-decene.

Preferably, the oil prepared according to the invention may also comprise at least one other saturated oligomer of 1-decene selected from among 9-methyl-nonadecane and 9-methyl-11,13-dioctyl-tricosane.

Moreover, the oil prepared according to the invention may also comprise other oligomers of 1-decene of greater sizes.

Particularly advantageously, the method according to the invention allows preparation of an oil comprising
from 51 to 99.9% by weight, preferably from 70 to 90% by weight, of a 1-decene trimer of formula (I) and
from 0.1 to 49% by weight, preferably from 10 to 30% by weight, of at least one other saturated trimer of 1-decene.

Also advantageously, the method according to the invention allows preparation of an oil comprising
from 51 to 99.6% by weight of a 1-decene trimer of formula (I);
from 0.1 to 1% by weight of at least one saturated dimer of 1-decene, for example 9-methyl-nonadecane;
from 0.1 to 25% by weight of at least one other saturated trimer of 1-decene;
from 0.1 to 20% by weight of at least one saturated tetramer or 1-decene, for example 9-methyl-11,13-dioctyl-tricosane;
from 0.1 to 1.5% by weight of at least one saturated pentamer of 1-decene.

The method according to the invention allows preparation of an oil which may be used as a base oil or as a lubricating base oil. This use therefore relates to a low viscosity oil comprising more than 50% by weight of 9-methyl-11-octyl-henicosane prepared according to the method of the invention.

The method according to the invention allows preparation of an oil which may also be used for improving the Fuel Eco (FE) of a lubricant, in order to reduce the consumption of fuel of an engine or further for reducing the fuel consumption of a vehicle engine.

The method according to the invention allows preparation of an oil which may be incorporated to a lubricant composition. This lubricant composition therefore comprises a low viscosity oil comprising more than 50% by weight of 9-methyl-11-octyl-henicosane prepared according to the method of the invention.

Advantageously, this lubricant composition may comprise at least 10% by weight or at least 20% by weight of an oil prepared according to the invention. Also advantageously, this lubricant composition may comprise at least 30, 40, 50 or 60% by weight of an oil prepared according to the invention.

Also advantageously, this lubricant composition may comprise an oil prepared according to the invention and at least one other base oil. It may also comprise an oil prepared according to the invention and at least one additive or else one oil prepared according to the invention, at least one other base oil and at least one additive.

As another base oil combined with the oil prepared according to the invention, this lubricant composition may comprise an oil selected from among an oil of group III, an oil of group IV.

The lubricant composition is particularly advantageous so as to be used as a high performance lubricant for lubrication in the fields of engines, gears, braking, hydraulic fluids, coolants, greases.

This lubricant composition may also give the possibility of improving the Fuel Eco (FE) of a lubricant, of reducing the fuel consumption of an engine or further reducing the fuel consumption of a vehicle engine.

The different aspects of the invention will be the subject of the examples which follow and which are provided as an illustration.

### EXAMPLES

An autoclave reactor equipped with a stirrer, a temperature control system and inlets for introducing nitrogen, hydrogen and 1-decene is used.

The 1-decene (product of the TCI or Acros company) is used at a purity of more than 94%. It is purified on molecular sieves of 3 Å and 13× (Sigma-Aldrich). Before use, the molecular sieves used are dried beforehand at 200° C. for 16 hours.

The products are characterized by ¹H NMR and by two-dimensional gas chromatography (GCxGC).

For NMR, the PAO samples were diluted in deuterated chloroform and the NMR spectra were produced at 300 K on Bruker 400 MHz spectrometers: ¹H, ¹³C, HMQC (heteronuclear multiple quantum coherence) and HMBC (heteronuclear multiple bond coherence).

The two-dimensional chromatography is applied in a continuous mode by means of two apolar and polar columns. The whole of the effluents stemming from the first column is separated in the second dimension. The separation of the compounds is governed by the volatility on the first column and by specific interactions (π-π type, dipolar interactions, etc) on the second dimension. Depending on their viscosity, the samples are generally diluted twice in heptane. The chromatographic conditions were optimized in order to be able to elute the PAOs prepared according to the invention. The samples were analyzed in GC×GC with cryogenic modulation (liquid nitrogen), programing the first oven from 45° C. (5 mins) up to 320° C. (20 mins) with a ramp of 3° C./min, programing of the secondary oven from 60° C. (5 mins) up to 330° C. (20 mins) with a ramp of 3° C./min and columns used according to the following operating conditions:
1^{st} dimension: HP1, 25 m, ID 0.32 mm, film thickness: 0.17 μm;
2^{nd} dimension: BPX-50, 1.5 m, ID 0.1 mm, film thickness: 0.1 μm;
injector: split 100:1, injected volume: 0.1 μl;
detector: FID, 320° C.;
hot jet temperature: 320° C.;
programing the cold jet from 80 to 5%;
modulation period: 4.8 s.

### Example 1

An 8 L autoclave reactor is used. Before its use, the reactor is dried at 130° C. with a nitrogen flow for one hour and then cooled to 110° C. Next, it is filled with 3,500 mL of 1-decene under a nitrogen flow. The temperature of the reactor is maintained at 110° C. and hydrogen (H₂) is introduced in a H₂/1-decene m/m ratio of 414 ppm.

The catalyst is the rac-ethylene bis(tetrahydroindenyl) zirconium dimethyl activated with dimethylanilinium tetrakis(perfluorophenyl)borate (DMAB) in a B/Zr molar ratio of 1.75. Triisobutyl aluminium (TiBAl) is used as a co-activator compound in an Al/Zr molar ratio of 200. It gives the possibility of trapping impurities present in the reactor. The oligomerization begins at the moment when the activated catalyst is introduced in a concentration of 17 μM relatively to the oligomerization solution.

After 120 mins, 5 mL of isopropanol is introduced in order to deactivate the catalyst. Next, one proceeds with hydrogenation of the reaction products by using a catalyst with palladium supported on alumina (5 g of palladium on gamma-alumina at 5% m/m with respect to alumina—the product Alfa Aesar) and hydrogen (H₂) at 20 bars, at a temperature of 100° C. for 240 mins.

The oligomerization products and the fraction of trimers comprising more than 50% by weight of 9-methyl-11-octyl-henicosane are then separated by distillation at reduced pressure (0.67 mbars (0.5 mmHg)) in two steps according to the ASTM D2892 standard and then according to the ASTM D5236 standard: (1) by means of a column with 15 theoretical plates for which the maximum temperature is 375° C. and then (2) by means of a column with 2 theoretical plates, the temperature of the vapors of which in the column head ranges from 375 to 445° C.

Distillation according to the ASTM D2892 standard allows separation of the products for which the boiling point is less than 375° C. The distillation according to the ASTM D5236 standard allows isolation of the products for which the boiling point ranges from 375 to 445° C.

The oil according to the invention obtained has a content of 9-methyl-11-octyl-henicosane equal to 71.4%.

This oil according to the invention comprising more than 50% by weight of 9-methyl-11-octyl-henicosane has a kinematic viscosity at 100° C., measured according to the ASTM D445 standard, of 3.448 mm²·s⁻¹. The viscosity index of this oil is 130. Its volatility measured according to the ASTM D6375 standard is 10.3% by mass and its dynamic viscosity (CCS) at −35° C., measured according to the ASTM D5293 standard, is 780 mPa·s. Its average molecular mass is 372 g/mol.

The characteristics of the oil according to the invention gives the possibility of obtaining excellent lubricant, rheological and oxidation resistance properties as well as Fuel Eco properties.

### Example 2

One proceeds in an identical way with example 1 for oligomerization of 1-decene.

The oligomerization products and the fraction of trimers comprising more than 50% by weight of 9-methyl-11-octyl-henicosane are then separated by distillation at reduced pressure (0.67 mbars (0.5 mmHg)) in two steps according to the ASTM D2892 standard and then according to the ASTM D5236 standard: (1) by means of a column with 15 theoretical plates for which the maximum temperature is 375° C. and then (2) by means of a column with 2 theoretical plates for which the temperature of the vapors in the column head ranges from 445 to 465° C.

The distillation according to the ASTM D2892 standard allows separation of the products for which the boiling point is less than 375° C. The distillation according to the ASTM D5236 standard allows isolation of the products for which the boiling point ranges from 445 to 465° C.

The obtained oil according to the invention has a content of 9-methyl-11-octyl-henicosane equal to 65.7%.

This oil according to the invention comprising more than 50% by weight of 9-methyl-11-octyl-henicosane has a kinematic viscosity at 100° C., measured according to the ASTM D445 standard, of 3.640 mm²·s⁻¹. The viscosity index of this oil is 132. Its volatility measured according to the ASTM D6375 standard is 9.1% by mass and its dynamic viscosity (CCS) at −35° C., as measured according to the ASTM D5293 standard, is 890 mPa·s. Its average molecular mass is 383 g/mol.

Again, the characteristics of this oil according to the invention give the possibility of obtaining excellent lubricant, rheological and oxidation resistance as well as Fuel Eco properties.

### Example 3

One proceeds in an identical way with example 1 in order to prepare a first fraction of oil according to the invention. One proceeds in an identical way with example 2 for preparing a second oil fraction according to the invention. Both fractions are then collected.

Next, one proceeds with the final hydrogenation by using a palladium catalyst (0.5% m/m relatively to H₂) supported on alumina (5 g of palladium on gamma-alumina at 5% m/m relatively to alumina—product Alfa Aesar) and hydrogen (H₂) at 20 bars, at a temperature of 90° C. for 240 mins.

The obtained oil according to the invention has a content of 9-methyl-11-octyl-henicosane equal to 74.7%.

This oil according to the invention comprising more than 50% by weight of 9-methyl-11-octyl-henicosane has a kinematic viscosity at 100° C., measured according to the ASTM D445 standard, of 3.569 mm²·s⁻¹. The viscosity index of this oil is 130. Its volatility measured according to the ASTM D6375 standard is 10.3% by mass and its dynamic viscosity (CCS) at −35° C., measured according to the ASTM D5293 standard is 720 mPa·s. Its average molecular mass is 378 g/mol.

Again, the characteristics of the oil according to the invention give the possibility of obtaining excellent lubricant, rheological and oxidation resistance as well as Fuel Eco properties.

### Comparative Example 1

Measurements and identical characterizations were carried out from of a reference commercial oil. This is a PAO oil (the product ExxonMobil Spectrasyn Plus 3.6) prepared from olefins by acid catalysis.

This reference PAO oil has a kinematic viscosity at 100° C., measured according to the ASTM D445 standard, of 3.671 mm²·s⁻¹. Its viscosity index is 118. Its volatility measured according to the ASTM D6375 standard is 14.3% by mass and its dynamic viscosity (CCS) at −35° C., as measured according to the ASTM D5293 standard is 1,100 mPa·s. Its average molecular mass is 374 g/mol.

Moreover, the specifications of this commercial oil are the following: kinematic viscosity at 100° C., measured according to the ASTM D445 standard, from 3.5 to 3.9 mm²·s⁻¹; volatility measured according to the ASTM D5800 standard of less than 17% by mass.

The method according to the invention therefore gives the possibility of preparing an oil for which the properties are equivalent or greater than the commercial PAO oils, in particular the viscosity index or the dynamic viscosity which are much better for the oils according to the invention.

1-18. (canceled)

19. A method for preparing an oil with a kinematic viscosity at 100° C., measured according to the ASTM D445 standard, ranging from 3 to 4 mm²·s⁻¹, comprising more than 50% by weight of a 1-decene trimer of formula (I), 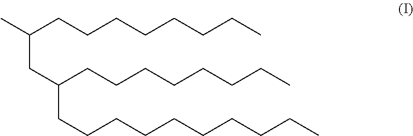 [SMILES: CCCCCCCCCCC(CCCCCCCC)CC(C)CCCCCCCC]
comprising
oligomerization of 1-decene in the presence of hydrogen (H₂), of a metallocene catalyst and of an activator compound or in the presence of hydrogen (H₂), of a metallocene catalyst, of an activator compound and of a co-activator compound;
catalytic hydrogenation of the oligomerization products in the presence of hydrogen (H₂) and of a hydrogenation catalyst;
separation by distillation at reduced pressure of the fraction of trimers comprising more than 50% by weight of the 1-decene trimer of formula (I).

20. The method according to claim 19 comprising a final hydrogenation step of the fraction of trimers comprising more than 50% by weight of the 1-decene trimer of formula (I), in the presence of hydrogen (H₂) and of a hydrogenation catalyst.

21. The method according to claim 19 comprising the recycling of the fraction of 1-decene dimers (for example 9-methyl-nonadecane), separated by distillation at reduced pressure and oligomerization of this fraction of this recycled fraction of 1-decene dimers with 1-decene, in the presence of hydrogen (H₂), of a metallocene catalyst and of an activator compound or in the presence of hydrogen (H₂) of a metallocene catalyst, of an activator compound and of a co-activator compound.

22. The method according to claim 19 comprising the deactivation of the catalyst after oligomerization of 1-decene and after catalytic hydrogenation of the oligomerization products.

23. The method according to claim 19 comprising the preliminary preparation of 1-decene by catalytic oligomerization of ethylene.

24. The method according to claim 19 wherein the oligomerization of 1-decene is achieved in the presence of hydrogen (H₂), of a metallocene catalyst, of an activator compound and of a co-activator compound.

25. The method according to claim 19 wherein the metallocene catalyst is a racemic compound of formula (II)
L(Q¹)(Q²)MR¹R² (II)
wherein
M represents a transition metal selected from among titanium, zirconium, hafnium, and vanadium or represents zirconium;
Q¹ and Q², either substituted or non-substituted, independently represent a tetrahydroindenyl cyclic group or Q¹ and Q² independently represent a tetrahydroindenyl cyclic group and are bound in order to form a polycyclic structure;
L represents a divalent C₁-C₂₀ alkyl group bridging Q¹ and Q² or L represents a group selected from among methylene (—CH₂—), ethylene (—CH₂—CH₂—), methylmethylene (—CH(CH₃)—), 1-methyl-ethylene (—CH(CH₃)—CH₂—), n-propylene (—CH₂—CH₂—CH₂—), 2-methylpropylene (—CH₂—CH(CH₃)—CH₂—), 3-methylpropylene (—CH₂—CH₂—CH(CH₃)—), n-butylene (—CH₂—CH₂—CH₂—CH₂—), 2-methylbutylene (—CH₂—CH(CH₃)—CH₂—CH₂—), 4-methylbutylene (—CH₂—CH₂—CH₂—CH(CH₃)—), pentylene and isomers thereof, hexylene and isomers thereof, heptylene and isomers thereof, octylene and isomers thereof, nonylene and isomers thereof, decylene and isomers thereof, undecylene and isomers thereof, dodecylene and isomers thereof;
R¹ and R², either substituted or non-substituted, independently represent an atom or a group selected from among hydrogen, halogens, and alkyls; or R¹ and R² form with M a metallocycle comprising from 3 to 20 carbon atoms.

26. The method according to claim 19 wherein the metallocene catalyst is selected from among rac-ethylene bis(tetrahydroindenyl) zirconium dimethyl and rac-ethylene bis(tetrahydroindenyl)zirconium dichloride.

27. The method according to claim 19 wherein the oligomerization of 1-decene is achieved
in a period ranging from 2 to 300 mins or from 5 to 180 mins or from 30 to 140 mins; or
in the presence of hydrogen (H₂) at a partial pressure ranging from 0.1 to 20 bars or from 1 to 6 bars; or
in a hydrogen/1-decene mass ratio greater than 100 ppm or less than 600 ppm or comprised between 100 and 600 ppm; or
at a temperature ranging from 50 to 200° C. or from 70 to 160° C. or from 80 to 150° C. or from 90 to 140° C. or from 100 to 130° C.; or
in a solvent selected from among a linear or branched hydrocarbon, a cyclic or non-cyclic hydrocarbon, an alkylated aromatic compound and mixtures thereof or in a solvent selected from among butanes, pentanes, hexanes, heptanes, octanes, cyclopentane, cyclohexane, methylcyclopentane, methylcyclohexane, methylcycloheptane, toluene, xylene and mixtures thereof.

28. The method according to claim 19 wherein
the activator compound is selected from among an ionic activator and an oligomeric compound comprising residues of formula —Al(R)—O— wherein R represent independently a cyclic or linear C₁-C₂₀ alkyl group; or
the activator compound is selected from among methylalumoxane, modified methylalumoxane, ethylalumoxane, isobutylalumoxane and mixtures thereof; or
the activator compound is selected from among dimethylanilinium tetrakis(perfluorophenyl)borate (DMAB), triphenylcarbonium tetrakis(perfluorophenyl)borate, dimethylanilinium tetrakis(perfluorophenyl)aluminate and mixtures thereof.

29. The method according to claim 19 wherein
the activator compound is an ionic activator and the co-activator compound is a trialkylaluminium derivative; or
the activator compound is an ionic activator and the co-activator compound is a compound selected from among tri-ethyl aluminium (TEAL), tri-iso-butyl aluminium (TIBAL), tri-methyl aluminium (TMA), methyl-methyl-ethyl aluminium (MMEAL) and tri-n-octyl aluminium.

30. The method according to claim 19 comprising deactivation of the catalyst, is carried out by action of air or by action of water or by means of at least one alcohol or a deactivation agent solution.

31. The method according to claim 19 wherein, during the catalytic hydrogenation of the oligomerization products,
the hydrogen pressure (H₂) ranges from 5 to 50 bars or from 10 to 40 bars or from 15 to 25 bars; or
the hydrogenation catalyst is selected from a derivative of palladium, a derivative of the supported palladium, a derivative of the supported palladium on alumina (for example gamma-alumina), a derivative of nickel, a derivative of supported nickel, a derivative of nickel supported on kieselguhr, a derivative of platinum, a derivative of supported platinum, a cobalt-molybdenum derivative, a supported cobalt-molybdenum derivative.

32. The method according to claim 19 comprising the final hydrogenation of the majority fraction by weight of the 1-decene trimer of formula (I), achieved
at a hydrogen pressure (H₂) ranging from 5 to 50 bars or from 10 to 40 bars or from 15 to 25 bars; or
within a period comprised between 2 and 600 mins or between 30 and 300 mins; or
at a temperature ranging from 50 to 200° C. or from 60 to 150° C. or from 70 to 140° C. or from 80 to 120° C.; or
in the presence of a hydrogenation catalyst selected from a derivative of palladium, a derivative of supported palladium, a derivative of supported palladium on alumina (for example gamma-alumina), a derivative of nickel, a derivative of supported nickel, a derivative of supported nickel on kieselguhr, a derivative of platinum, a derivative of supported platinum, a cobalt-molybdenum derivative, a supported cobalt-molybdenum derivative.

33. The method according to claim 19 for preparing an oil comprising
from 60 to 90% by weight of the 1-decene trimer of formula (I) or from 70 to 90% by weight of the 1-decene trimer of formula (I); or
at least 65% by weight of the 1-decene trimer of formula (I) or at least 70% by weight of the 1-decene trimer of formula (I) or at least 80% by weight of the 1-decene trimer of formula (I) or at least 90% by weight of the 1-decene trimer of formula (I).

34. The method according to claim 19, for preparing an oil also comprising at least one other saturated oligomer of 1-decene selected from among
the other trimers of 1-decene; or
the dimers of 1-decene, the other trimers of 1-decene, the tetramers of 1-decene, the pentamers of 1-decene; or
9-methyl-nonadecane and 9-methyl-11,13-dioctyl-tricosane.

35. The method according to claim 19, for preparing an oil comprising
from 51 to 99.9% by weight of the 1-decene trimer of formula (I) and from 0.1 to 49% by weight of at least one other saturated trimer of 1-decene; or
from 70 to 90% by weight of the 1-decene trimer of formula (I) and from 10 to 30% by weight of at least one other saturated trimer of 1-decene; or
from 51 to 99.6% by weight of the 1-decene trimer of formula (I);
from 0.1 to 1% by weight of at least one saturated dimer of 1-decene (for example 9-methyl-nonadecane);
from 0.1 to 25% by weight of at least one other saturated trimer of 1-decene;
from 0.1 to 20% by weight of at least one saturated tetramer of 1-decene (for example 9-methyl-11,13-dioctyl-tricosane);
from 0.1 to 1.5% by weight of at least one saturated pentamer of 1-decene.

36. The method according to claim 19 for preparing an oil comprising more than 50% by weight of 9-methyl-11-octyl-henicosane for which
(a) the kinematic viscosity at 100° C., measured according to the ASTM D445 standard ranges from 3.2 to 3.8 mm²·s⁻¹ or is 3.5 mm²·s⁻¹; or for which
(b) the viscosity index is greater than 120 or greater than or equal to 130 or is comprised between 120 and 140 or between 125 and 135; or for which
(c) the volatility measured according to the ASTM D6375 standard is less than 10.8% by mass or less than 10.5% by mass; or for which
(d) the dynamic viscosity (CCS) at −35° C., measured according to the ASTM D5293 standard is less than 900 mPa·s or less than 800 mPa·s.

37. The method of claim 25, wherein R¹ and R² independently represent a halogen selected from a group consisting of Cl and I.

38. The method of claim 25, wherein R¹ and R² independently represent an alkyl selected from a group consisting of Me, Et, nPr and iPr, alkenyl, alkynyl, halogenoalkyl, halogenoalkenyl, halogenoalkynyl, silylalkyl, silylalkenyls, silylalkynyls, germylalkyl, germylalkenyl, and germylalkynyl.
